(19) United States
(12) Patent Application Publication
(10) Pub. No.: US 2024/0277343 A1
(43) Pub. Date: Aug. 22, 2024

(54) SYSTEMS FOR CLOT LENGTH AND PROPERTY CHARACTERIZATION

(71) Applicant: KONINKLIJKE PHILIPS N.V., EINDHOVEN (NL)

(72) Inventors: Alexandru PATRICIU, BELMONT, MA (US); Marcin A. BALICKI, CAMBRIDGE, MA (US); Roland Wilhelmus Maria BULLENS, MIERLO (NL); Matthew MAGEE, COLORADO SPRINGS, CO (US); James David CEZO, COLORADO SPRINGS, CO (US); Michael ANDERSON, COLORADO SPRINGS, CO (US)

(21) Appl. No.: 18/569,037

(22) PCT Filed: Jun. 15, 2022
(86) PCT No.: PCT/EP2022/066274

§ 371 Date: Dec. 11, 2023

Related U.S. Application Data
(60) Provisional application No. 63/214,289, filed on Jun. 24, 2021.

(51) Int. Cl. A61B 17/12; A61B 6/12; A61B 17/22; A61B 34/20; A61B 34/30; A61B 90/00

(52) U.S. Cl. CPC A61B 17/12172; A61B 6/12; A61B 17/1204; A61B 34/20; A61B 34/30; A61B 2017/22038; A61B 2034/2065; A61B 2034/301; A61B 2090/3966

(57) ABSTRACT

A thrombectomy device (10) includes a retrieval device (12) configured to deploy and subsequently retrieve an associated tethered self-expanding stent (2), the retrieval device including a guidewire (14) having a tip (13) that is radiopaque. At least one electronic processor (20) is programmed to: receive a time sequence of images (35) of extension of the guidewire through a clot during a thrombectomy procedure being performed using the thrombectomy device: determine a geometric change of the tip of the guidewire; identify a completion of the extension of the guidewire through the clot based on the geometric change of the tip of the guidewire; and respond to the identification of the completion of the extension of the guidewire through the clot by: outputting an indication (36) that the guidewire is extended completely through the clot; and/or controlling a robot (16) to stop the extension of the guidewire through the clot.

### FIELD

The following relates generally to the catheter arts, mechanical thrombectomy arts, imaging arts, clot retrieval arts, and related arts.

### BACKGROUND

Vascular therapy (e.g., thrombectomy, atherectomy, and so forth) devices are medical devices designed to remove or modify tissue or material from inside a diseased vessel (e.g., an artery, a vein, etc.). In particular, mechanical thrombectomy is an effective treatment for ischemic strokes by direct removal of arterial brain clots. In such procedures, a removal device is deployed through the clot. Proper device selection and placement is important for procedure success. Typically, a stent retriever can be used for such procedures.

A mechanical thrombectomy typically includes the following steps: (1) access the carotid artery proximal to the thrombus with a balloon guide catheter; (2) access a thrombus or clot site using a micro-catheter/micro-guidewire combination through the balloon catheter; (3) advance the micro-guidewire past the thrombus; (4) advance the micro-catheter over the micro-guidewire past the thrombus; (5) retract the micro-guidewire and replace with stent retriever; (6) deploy the stent retriever; (7) wait for stent integration with the clot; (7) deploy the balloon of the balloon guide catheter; and (8) retrieve the thrombus by retracting together the stent retriever and microcatheter into the balloon guide catheter.

The following discloses certain improvements to overcome these problems and others.

### SUMMARY

In some embodiments disclosed herein, a thrombectomy device includes a retrieval device configured to deploy and subsequently retrieve an associated tethered self-expanding stent. The retrieval device includes a guidewire having a tip that is radiopaque. At least one electronic processor is programmed to: receive a time sequence of images of extension of the guidewire through a clot during a thrombectomy procedure being performed using the thrombectomy device; perform image analysis on the images of the time sequence of images to determine a geometric change of the tip of the guidewire; identify a completion of the extension of the guidewire through the clot based on the geometric change of the tip of the guidewire; and respond to the identification of the completion of the extension of the guidewire through the clot by: outputting an indication that the guidewire is extended completely through the clot; and/or controlling a robot to stop the extension of the guidewire through the clot.

In some embodiments disclosed herein, a thrombectomy device includes a microcatheter; a self-expanding stent tethered to the microcatheter and configured to be compacted into the microcatheter; and a guidewire having a tip. The tip of the guidewire is radiopaque and/or includes a sensor measuring a geometry of the tip.

In some embodiments disclosed herein, a vascular therapy method includes: receiving a time sequence of images of extension of a guidewire through a clot during a thrombectomy procedure being performed using a thrombectomy device; performing image analysis on the images of the time sequence of images to determine a geometric change of a tip of the guidewire; identifying a completion of the extension of the guidewire through the clot based on the geometric change of the tip of the guidewire; and responding to the identification of the completion of the extension of the guidewire through the clot by: outputting an indication that the guidewire is extended completely through the clot; and/or controlling a robot to stop the extension of the guidewire through the clot.

One advantage resides in providing a stent retrieval device that allows the stent to withdraw a clot in a single attempt.

Another advantage resides in providing improved control of a stent retrieval process to reduce likelihood of a clot breaking into smaller fragments.

Another advantage resides in providing mechanical thrombectomy with improved timing of the stent retrieval to ensure full integration with the clot while avoiding unnecessary delay in initiating the stent retrieval.

Another advantage resides in providing feedback during stent retrieval to determine a geometric change in a tip of the guidewire which the stent is tethered to.

Another advantage resides in controlling a speed of a robotic member withdrawing a guidewire after stent delivery within a clot in a patient.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIGS. 1A, 1B, 1C, and 1D diagrammatically illustrates a vascular therapy device performing a guidewire extension sequence (FIGS. 1A, 1B, and 1C) followed by a stent deployment operation (FIG. 1D) in accordance with the present disclosure.

FIG. 2 diagrammatically illustrates a method of performing a vascular therapy method using the device of FIG. 1.

### DETAILED DESCRIPTION

In step (2) of a mechanical thrombectomy (i.e., insertion of the guidewire through the clot), the guidewire must be advanced until it passes the clot, so that the stent retriever is deployed fully into the clot. It is important that the tip of the guide wire is deployed past the clot such that the subsequently deployed stent retriever will retrieve the entire clot. but the guide wire should not be deployed too far past the clot such that it does not damage healthy blood vessel or lead to the subsequently deployed stent retriever being placed past the clot. In addition, accurately determination of the length of the clot can be used to select the correct stent retriever type and size. Therefore, it is advantageous to detect the length of the clot.

The following relates to mechanical thrombectomy, which is a technique in which a self-expanding (e.g., nitinol) stent that is tied to a retrieval line is deployed into a clot. The stent expands and integrates with the clot, and then the stent with the integrated clot is pulled back into the catheter using the retrieval line to remove the clot from the bloodstream.

In this procedure, it is important to capture the entire clot with the stent. If the stent is deployed only partway into the clot, then the clot is likely to break apart during the stent retrieval step, leading to loosened clot fragments entering the bloodstream with potentially significant adverse clinical impact.

Presently, the stent deployment entails extending a guidewire through the expected length of the clot, then moving a microcatheter along the guidewire, withdrawing the guidewire from the microcatheter and moving the stent into the microcatheter, then withdrawing the microcatheter to deploy the stent. Radiopaque markers are generally included on the stent, so it is visible in fluoroscope monitoring images. However, the clot is at best weakly visible in the fluoroscope imaging and may not be visible at all, and the guidewire is also weakly visible or invisible in the fluoroscope monitoring images—hence, the operator has no assurance that the guidewire has passed completely through the clot such that the subsequently deployed stent fully captures the entire length of the clot.

The approaches disclosed herein take advantage of the fact that the tip of the guidewire has reduced stiffness to facilitate pushing the guidewire through the clot. Consequently, the guidewire tip tends to curl around to form a knuckle or partial loop. Prior to entering the clot, the guidewire tip partial loop is expanded or uncurled, due to the relatively large diameter of the blood vessel lumen. When the tip enters the constrictive clot, the partial loop is compressed laterally by the clot which has a narrowed lumen compared with the lumen of the unobstructed blood vessel. The tip stays compressed while passing through the clot. When the tip of the guidewire reaches the end of the clot and passes back into healthy vessel, the partial loop at the tip expands again into the larger-diameter blood vessel lumen.

Recognizing the foregoing, the following discloses making the guidewire tip radiopaque by adding a radiopaque wire (or making the tip of a radiopaque wire), or adding a radiopaque coating, or adding radiopaque markers, and/or so forth. The partial loop at the tip (or, more generally, the geometric shape of the tip) is then observable in the fluoroscope images, and the compression of the partial loop as it passes through the clot and its subsequent expansion when it passes out of the clot can be observed in the fluoroscope image as geometric changes of the tip of the guidewire (e.g., its compression when entering the clot, and its subsequent decompression or unfurling upon exiting the clot).

In some embodiments disclosed herein, various image processing techniques for detecting the compression and subsequent expansion of the partial loop are implemented. One complication in this regard is that the guidewire is sometimes rotated, e.g., at around 0.5-1.0 Hz, during the insertion to facilitate passage through the clot, while the real-time fluoroscope imaging operates at a typical frame rate of around 5-10 fps in some cases. The fluoroscope images are two-dimensional (2D) projection images, and the disclosed signal processing techniques take the rotation of the tip into account in assessing the amount of compression or expansion of the partial loop at the guidewire tip. For example, at 5 fps and a 1 Hz rotation, each complete 360º rotation of the tip is captured by five image frames. Of these five image frames, the one showing the largest (projection of) the partial loop at the tip can be taken as the tip geometry. If the advancement rate of the tip through the clot is reasonably slow then this value can be further averaged over several rotations in a sliding window-type approach.

The disclosed approach can include both manual and robotic embodiments. In manual embodiments, the operator can directly observe the tip, possibly with a reference image of the tip taken prior to entry into the clot also shown for comparison. Optionally, the image processing can automatically detect expansion of the partial loop as it exits the clot and a light and/or audio signal is given to indicate when the microcatheter tip has passed completely through the clot. In a robotic approach, the insertion of the guidewire is performed automatically, and the robot stops insertion when the partial loop expansion is detected.

As a further option, the length of the clot can be determined by the following approach. Assuming a constant rate R of advancement of the guidewire (for example, in millimeters-per-second, mm/s), the time t_{entrance} is stored at which the tip geometry compresses indicating entry of the guidewire tip into the clot. Likewise, the time tₑₓᵢₜ is stored at which the tip geometry unfurls or expands indicating exit of the guidewire tip from the clot. Then the length L_{clot} of the clot can be estimated as L_{clot}=R×(tₑₓᵢₜ−t_{entrance}). Alternatively, if the guidewire manipulator records an extension length L_{wire}(t) of the guidewire as a function of time (for example, based on rotary encoder measurement of the rotation of a gear used in extending the guidewire), then the length of the clot can be estimated as L_{clot}=L_{wire} (tₑₓᵢₜ)−L_{wire} (t_{entrance}). This latter approach will work even if the rate R of advancement of the guidewire is not constant.

While described in conjunction with mechanical thrombectomy, more generally the disclosed technique for assessing passage of a guidewire through a clot can be usefully applied for any vascular treatment procedure that includes the operation of inserting a guidewire through a clot. For example, in some types of laser atherectomy, a guidewire is inserted through the clot which is then used to guide the catheter bearing the laser aperture to the clot. As another example, the atherectomy may employ a catheter bearing a mechanical (e.g. rotary) cutter. As yet another example, in some types of balloon angioplasty procedures, a guidewire is inserted through the clot which is then used to guide the catheter bearing a deflated balloon to the clot, which is then inflated to mechanically remodel the clot. These are merely further illustrative examples.

In a variant embodiment, a fiber optical sensor, piezoelectric bend sensor, or other sensor directly measures the contraction/expansion of the partial loop at the guidewire tip.

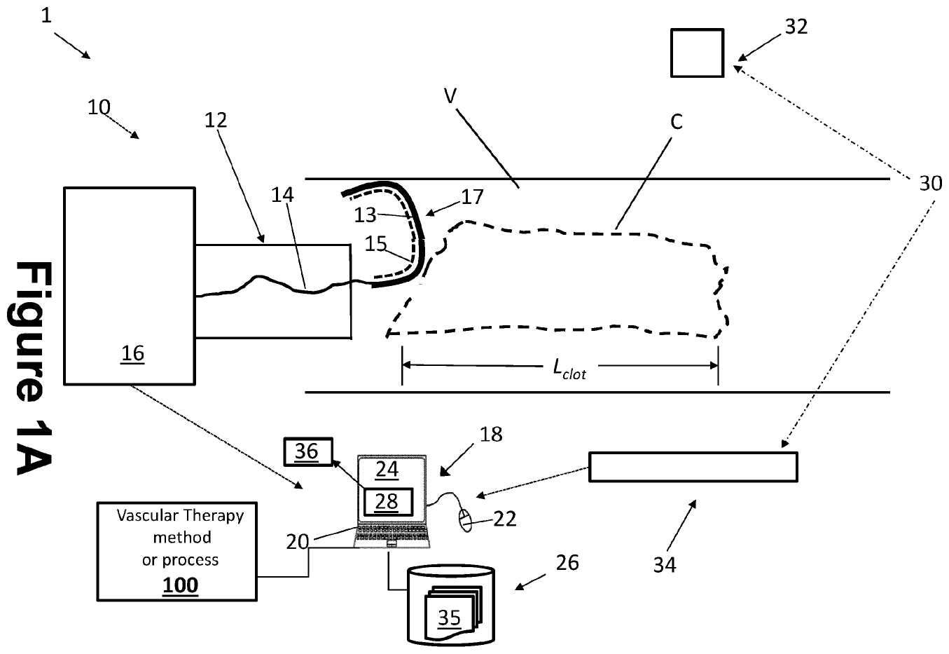

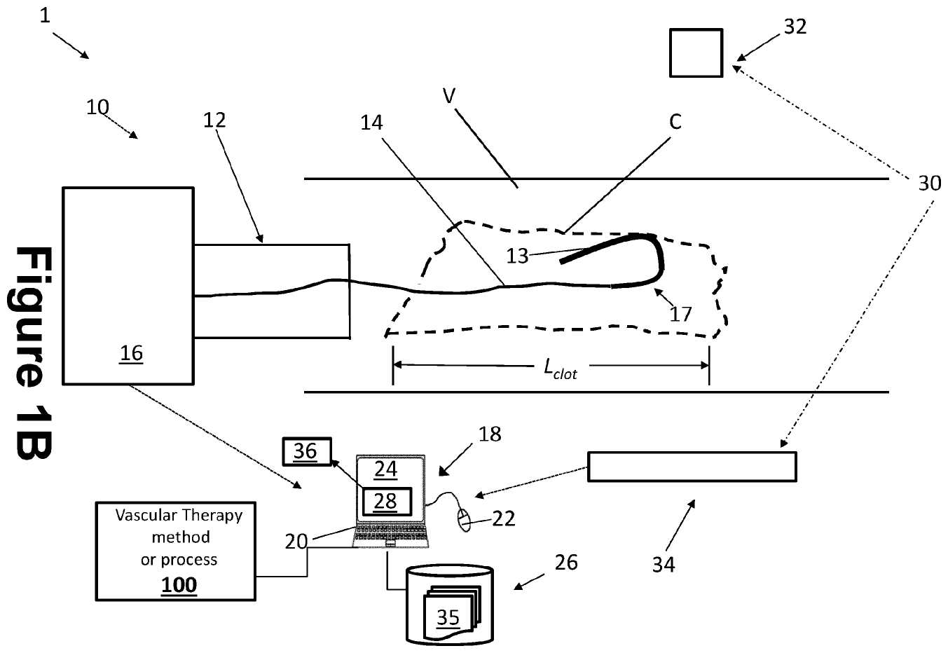

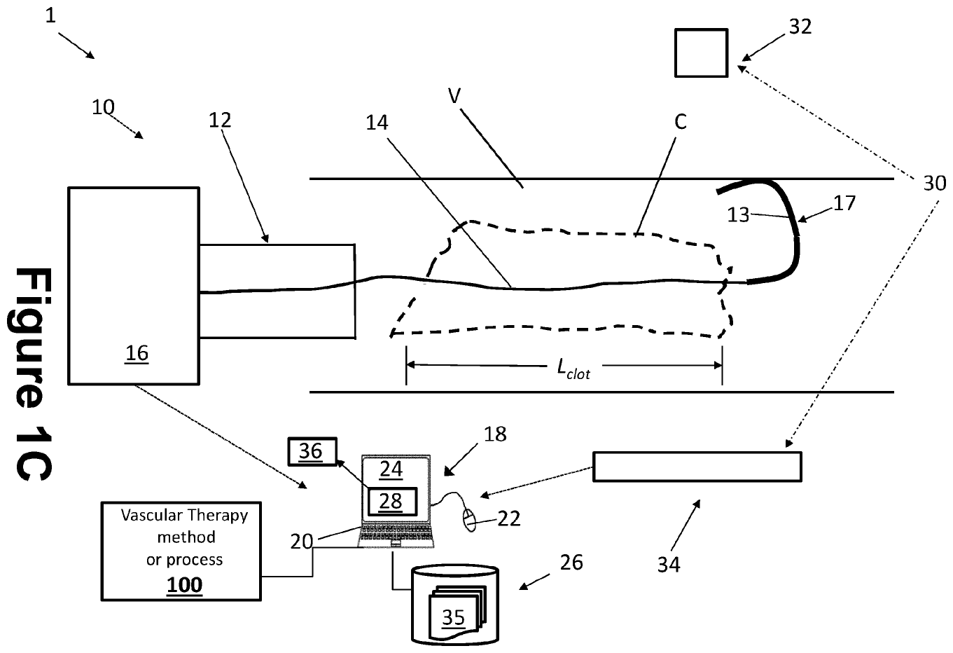

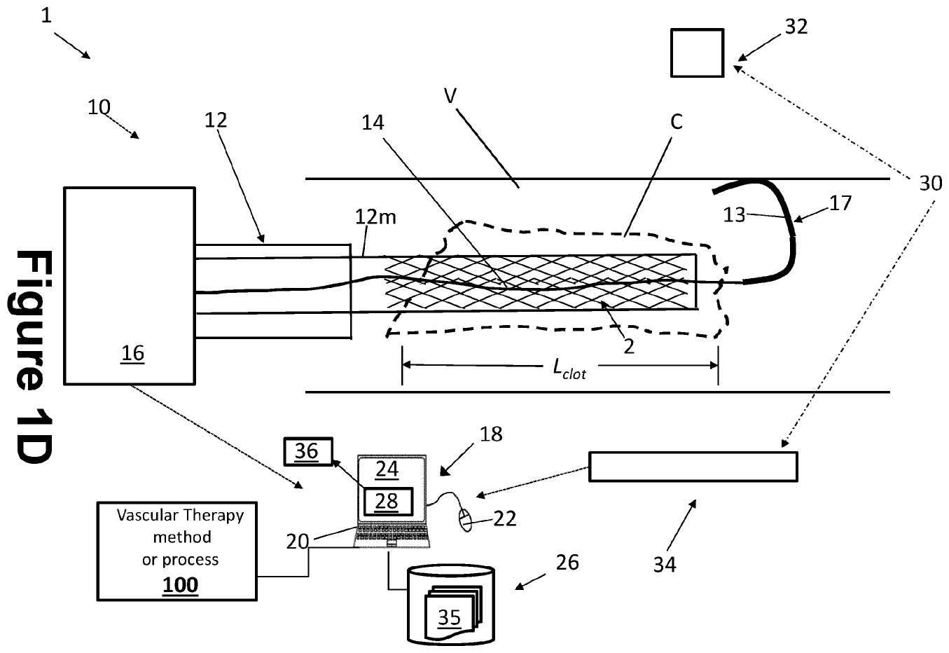

With reference to FIGS. 1A, 1B, 1C, and 1D, a guidewire extension sequence (FIGS. 1A, 1B, and 1C) followed by stent deployment (FIG. 1D) performed by an illustrative vascular therapy (i.e., thrombectomy or atherectomy) apparatus 1 is diagrammatically shown. As shown in FIGS. 1A-1D, the apparatus 1 includes a therapy device 10 for delivering and retrieving a self-expanding vascular therapy device 2 (shown only in FIG. 1D, e.g., a self-expanding stent, a self-expanding filter, and so forth) into a blood vessel. The therapy device 10 includes a catheter 12 configured to deploy, and subsequently receive, the self-expanding stent 2 in a clot C (diagrammatically shown in FIGS. 1A-1D with dashed lines) in a blood vessel V of a patient. In some embodiments, a microcatheter 12m (shown only in FIG. 1D) is extended from the catheter 12 for the stent deployment, with the self-expanding stent 2 is compacted inside the microcatheter 12m as shown in FIG. 1D.

As shown in FIG. 1A, the guidewire 14 includes a tip 13 that is radiopaque, so that it is imaged in fluoroscopic imaging. To make the tip 13 radiopaque, the tip 13 may be made of a different material from the bulk of the guidewire 14, e.g. the tip 13 may comprise a short radiopaque wire (e.g., a platinum or Nitinol wire) that is metallurgically bonded (e.g., by welding) to the end of the guidewire 14. Alternatively, radiopaque markers (not shown; for example, platinum tungsten-filled polyurethane bands) may be disposed at intervals along the tip 13. In yet another approach, the tip 13 may comprise the same wire as the guidewire 14 (that is, the tip 13 may be the end of the wire making up the guidewire 14) and is coated with a radiopaque coating such as a tantalum coating. These are merely illustrative examples. As shown in FIGS. 1A-1D, the tip 13 typically bends around to form a partial loop 17.

In some embodiments, as shown in FIG. 1, the tip 13 of the guidewire 14 can include a sensor 15 attached thereto and configured to measure a geometry, such as contraction or an expansion, of the tip 15. (The sensor 15 is diagrammatically indicated only in FIG. 1A). As noted, the tip 13 of the guidewire 14 typically forms a loop or partial loop 17. The sensor 15 can be configured to measure a geometric change of the loop 17. In some embodiments, the sensor 15 can comprise a bend sensor, such as a piezoelectric bend sensor or a resistive coating that changes electrical resistivity as it is bent, or a fiber optic bend sensor. As the loop 17 being to lose light as it bends more sharply, a decrease in an intensity of light passing through the loop 17 by the sensor 15 is a measure of the amount of bend.

FIG. 1 also shows a robot 16 (diagrammatically shown in FIG. 1 as a box) operatively connected to the guidewire 14. The robot 16 is configured to control extension of the guidewire 14 and/or deployment and subsequent retrieval of the self-expanding stent 2 deployed in the clot C subsequently to the extension of the guidewire 14. FIGS. 1A-1D further shows an electronic processing device 18, such as a workstation computer, or more generally a computer which can be used to control the robot 16 to automatically perform the extension of the guidewire 14 through the clot C. The electronic processing device 18 may also include a server computer or a plurality of server computers, e.g., interconnected to form a server cluster, cloud computing resource, or so forth, to perform more complex computational tasks. The workstation 18 includes typical components, such as an electronic processor 20 (e.g., a microprocessor), at least one user input device (e.g., a mouse, a keyboard, a trackball, and/or the like) 22, and a display device 24 (e.g., an LCD display, plasma display, cathode ray tube display, and/or so forth). In some embodiments, the display device 24 can be a separate component from the workstation 18 or may include two or more display devices.

The electronic processor 20 is operatively connected with one or more non-transitory storage media 26. The non-transitory storage media 26 may, by way of non-limiting illustrative example, include one or more of a magnetic disk, RAID, or other magnetic storage medium; a solid-state drive, flash drive, electronically erasable read-only memory (EEROM) or other electronic memory; an optical disk or other optical storage; various combinations thereof; or so forth; and may be for example a network storage, an internal hard drive of the workstation 18, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a visualization of a graphical user interface (GUI) 28 for display on the display device 24.

FIGS. 1A-1D diagrammatically show a typical vascular therapy treatment suitably performed with the illustrative vascular therapy (i.e., thrombectomy or atherectomy) apparatus 1. In FIG. 1A, the guidewire 14 is being initially extended from the catheter 12, and the radiopaque tip 13 of the guidewire 14 has not yet entered the clot C. The partial loop 17 of the tip 13 is of large diameter due to the space available in the relatively large lumen of the blood vessel V. At a time T_{entrance}, the tip 13 enters the clot C, at which point the loop 17 is compressed to conform with the smaller diameter of the lumen of the clot C. FIG. 1B illustrates the continued extension of the guidewire 14 at a point where the tip 13 is within the clot C and therefore the partial loop 17 is compressed by the smaller lumen of the clot C. At a subsequent time tₑₓᵢₜ the tip 13 passes out of the clot C, at which it expands due to the larger diameter of the blood vessel V, as shown in FIG. 1C. Finally, FIG. 1D illustrates the microcatheter 12m with the self-expanding stent 2 compressed inside being extended along the guidewire 14 into the clot C. In a typical mechanical thrombectomy procedure, this is then followed by withdrawal of the microcatheter 12m to deploy the stent 2 in the clot C where the stent 2 expands and integrates into the clot C, after which the stent 2 is drawn back into the catheter 12 to remove the clot C from the blood vessel V (stent deployment and retrieval steps not depicted in the sequence of FIGS. 1A-1D).

FIG. 1D merely depicts one illustrative example of a vascular therapy that may be performed using the extended guidewire 14 shown in FIG. 1C. As another example, in an atherectomy procedure, a catheter bearing a laser aperture or mechanical cutter may be delivered along the extended guidewire 14 to the clot C, where the laser aperture or cutter is employed in cutting away the clot C. As yet another example, in a balloon angioplasty procedure, a catheter bearing a deflated balloon may be delivered along the extended guidewire 14 to the clot C, where the balloon is then inflated to mechanically remodel the clot C. These are merely further illustrative examples.

Optionally, the disclosed technique can also be leveraged to measure the length of the clot C. As the guidewire 14 is being extended over the ordered sequence shown in FIG. 1A, FIG. 1B, and finally FIG. 1C, it follows that tₑₓᵢₜ>t_{entrance}. If the guidewire 14 is advanced at a constant speed R then optionally the length L_{clot} of the clot C can be estimated as L_{clot}=R×(tₑₓᵢₜ−t_{entrance}). Alternatively, if the robot 16 records an extension length L_{wire}(t) of the guidewire 14 as a function of time (for example, based on rotary encoder measurement of the rotation of a gear used in extending the guidewire), then the length of the clot can be estimated as L_{clot}=L_{wire} (tₑₓᵢₜ)−L_{wire} (t_{entrance}). This latter approach will work even if the rate R of advancement of the guidewire is not constant.

In a manual embodiment, the robot 16 is suitably replaced by a manual mechanism for performing the guidewire extension. In this embodiment, the GUI 28 suitably presents an alert when at the time Tₑₓᵢₜ based on the expansion or unfurling (or other geometric change) of the loop 17 as the tip 13 exits the clot C as shown in FIG. 1D, with the geometric change of the tip being detected by image analysis of real-time fluoroscopy images as described below. Optionally, the GUI 28 may also initially present an alert at the time T_{entrance} when the tip 13 enters the clot C, based on image analysis detecting the compression of the loop 17 as the tip 13 enters the clot C (where the time t_{entrance} is between the timepoint shown in FIG. 1A and the timepoint shown in FIG. 1B). If the advancement of the guidewire 14 is done manually using (for example) a hand-operated geared mechanism with suitable rotary encoder or the like to measure guidewire extension, then optionally the clot length can be estimated as L_{clot}=L_{wire} (tₑₓᵢₜ)−L_{wire} (t_{entrance}) as described previously for the robotic embodiment. In yet another approach, if the guidewire 14 has tic marks, e.g. every 1 mm along the length of the guidewire 14, then the length of the clot can be estimated by the operator as the number of tic marks of wire extension between the time t_{entrance} indicated by the GUI 28 and the time tₑₓᵢₜ indicated by the GUI 28.

FIGS. 1A-1D also show an imaging device 30 configured to acquire a time sequence of images 35 of the extension of the guidewire 14. In particular, in the illustrative examples the imaging device 30 is a fluoroscopic imaging device (e.g., an X-ray imaging device, C-arm imaging device, a CT scanner, or so forth) and the radiopaque tip 13 of the guidewire 14 is visible under the fluoroscopic imaging, thereby allowing the time tₑₓᵢₜ when the tip 13 exits the clot C to be determined. Optionally, the time t_{entrance} when the tip 13 enters the clot C is also determined. The fluoroscopic imaging is real-time imaging, e.g. with images being acquired at a frame rate of 5-10 frames/second (i.e. 5-10 fps) in some nonlimiting illustrative embodiments. The imaging device 30 is in communication with the at least one electronic processor 20 of the electronic processing device 18. As shown in FIG. 1, the imaging device 30 comprises an X-ray imaging device including an X-ray source 32 and an X-ray detector 34, such as a C-arm imaging device; however, it will be appreciated that any suitable imaging device, such as ultrasound (US), computed tomography (CT), magnetic resonance imaging (MRI), nuclear imaging, or any other suitable imaging device may be used. For non-fluoroscopic imaging devices, the radiopaque tip 13 is suitably replaced by a tip of a type that is observable in the chosen imaging modality. For example, if MRI is used for monitoring, then the radiopaque tip 13 is suitably made of a material, or includes markers of a material, that provides good contrast in MRI images. The images 35 can be stored in the non-transitory storage media 26.

The at least one electronic processor 20 is configured as described above to perform a vascular therapy method or process 100. The non-transitory storage medium 26 stores instructions which are readable and executable by the at least one electronic processor 20 to perform disclosed operations including performing the vascular therapy method or process 100. In some examples, the method 100 may be performed at least in part by cloud processing.

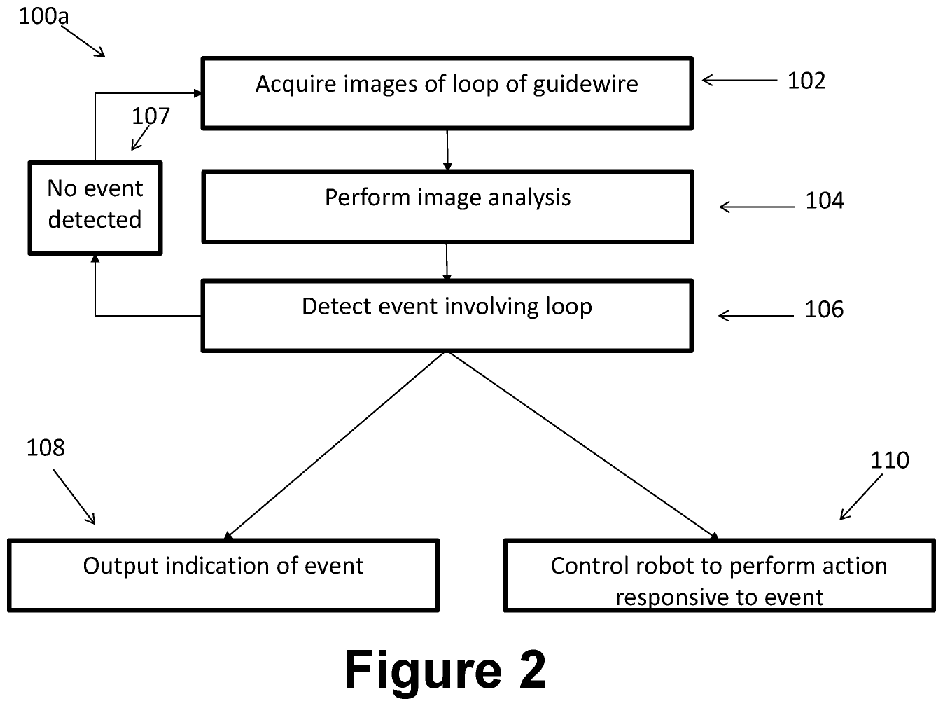

Referring to FIG. 2, and with continuing reference to FIG. 1, an illustrative embodiment of a guidewire extension phase 100a of the vascular therapy method 100 is diagrammatically shown as a flowchart. To begin the method 100a, the guidewire 14 is extended from the catheter 12 into the blood vessel V using the robot 16, or manually.

While the guidewire extension is in progress, at an operation 102, the imaging device 30 acquires a time sequence of images of the extension of the guidewire 14 through the clot C. The time sequence of images is then transferred to the electronic processing device 18.

At an operation 104, image analysis is performed on the images of the time sequence of images to determine a geometric change of the tip 13 (i.e., the loop 15) of the guidewire 14. In some embodiments, the image analysis is performed to determine that the geometric change in the loop 17 is that the loop 17 is compressing as it enters the clot C. In some embodiments, the image analysis is performed to determine that the geometric change in the loop 17 is that the loop 17 is expanding after completion of the extension of the guidewire 14 through the clot C. Typically, the loop 17 is compressed by tissue of the clot C as the guidewire 14 extends through the clot C (as shown in FIG. 3B). When the loop 17 is past the clot C (as shown in FIG. 3C), it is no longer compressed, and begins to expand. This expansion of the loop 17 indicates that the extension of the guidewire 14 is complete.

At an operation 106, if an event is identified during the guidewire extension based on the geometric change of the loop 17, then a response is performed based on the identification of the event at the operation 106; otherwise, flow passes 107 back in iterative fashion to operation 102 to continue acquiring images until an iteration of the operation 106 identifies an event. In some embodiments, at an operation 108, the response includes outputting an indication 36 of the event. To do so, the indication 36 can be a visual message or warning displayed via the GUI 28 on the display device 24, an audible indication output by the electronic processing device 18, and so forth. In some examples, the indication 36 can comprises messages such as “indication that the guidewire is entering the clot”, or “indication that the guidewire is extended completely through the clot”, or “ready for retrieval device deployment”, and so forth.

In some embodiments, at an operation 110, the response includes controlling the robot 16 to perform an action in response to the event (e.g., withdraw the guidewire 14 via the retrieval device 12, slow a speed of withdrawal of the guidewire 14, and so forth).

In one example, the event is identified prior to retrieval of the guidewire 14 from the clot C. The event comprises a cessation of expansion of the loop 17. Responsive to this identification, the robot 16 is controlled to initiate the next step in the vascular procedure, such as starting the stent deployment process of a mechanical thrombectomy procedure (see FIG. 1D).

In other embodiments, both operations 108 and 110 can be performed. For example, once an indication 36 is output at the operation 108, the robot 16 can be controlled to initiate stent deployment at the operation 110. Furthermore, although not shown in FIG. 2, after detection of the tip 13 entering the clot C and recordation of the corresponding time t_{entrance} and subsequent detection of the tip 13 exiting the clot C and recordation of the corresponding time tₑₓᵢₜ, the length L_{clot} of the clot C can be estimated using one of the approaches previously described.

### EXAMPLE

With continuing references to FIGS. 1 and 2, the operations of the apparatus 1 are described in more detail.

During the operation 102, in which the images 35 are acquired by the imaging device 30, the guidewire 14 can be rotated prior to reaching the clot C, and images 35 can be acquired. The shape of the tip 13 of the guidewire 14 can be detected from these images 35 using standard image processing techniques or AI methods such as U-net for segmentation and wire profile parametrization as outlined in Ambrosini et al (e.g., Ambrosini, Pierre, et al. “Fully automatic and real-time catheter segmentation in X-ray fluoroscopy.” International Conference on Medical Image Computing and Computer-Assisted Intervention. Springer, Cham, 2017.). The tip 13 will be shown in the images 35 in a cyclical pattern due to the rotation of the guidewire 14. These images 36 can be stored (at least temporarily) in the non-transitory computer readable medium 26 of the electronic processing device 18.

The guidewire 14 can then be advanced through the clot C, and additional images 35 can be acquired. During the operation 104, the shape of the tip 13 (i.e., the loop 17) is detected in each image 35 and compared with the shape of the loop 17 in the previously-acquired images. Advancement of the guidewire 14 can be stopped when the shape of the loop 17 is close to one or the stored images 35.

The image comparison analysis operation can be performed with a single “previous” image 35 (i.e., before the guidewire 14 is inserted into the clot C), and a single “current” image 35 (i.e., while the guidewire 14 is in the clot C). For the “pre-clot” pattern, store the shape of the loop 17 can be stored with a maximum radius. For each acquired image 35, the current shape of the loop 17 is compared with the stored shape of the loop 17. If there is a description of the shape of the loop 17 in 2D given as a sequence of points, a rigid registration of the two sets of points can be implemented. If the shapes are similar, then the distance between the two shapes will be small after the registration operation. If the shapes are very different then the error will be large. Assuming that the pre-clot shape is given by the points pi; i=1 . . . n and the current shape is given by the points qi; i=1 . . . , n, then a transformation H can be found that minimizes the least squares difference between the two sets of points. Then, if the error (Err) is less than a predetermined threshold, the shapes are similar. Therefore, the guidewire 14 exited the clot C.

The error measure Err can be constructed in various ways. The example above computes the distance between points. Alternatively, the distance between points and segments, or points and curves can be computed. Furthermore, the points of the two shapes and the estimated shape can be considered to get a more refined match.

The shape of the loop 17 during clot traversing can also be used to discriminate between soft clots and hard clots. When traversing a soft clot, the loop 17 will have a lower curvature then when traversing a hard clot. Therefore, by using image processing algorithms that analyze images 35 to compute the curvature of the loop 17 and classify the clot stiffness.

In another embodiment, the image comparison analysis operation can be performed with multiple “previous” images 35 and multiple “current” images 35. In this embodiment, a sliding window of acquired images 35 (i.e., acquired shapes of the loop 17) is sued. The sequence of “current” loop shapes is compared with the sequence of “previous” images 35. A “global registration” is obtained by registering the current images 35 with the previous images 35.

A total error between the shapes of the loop 17 in the previous images 35 and the acquired images 35 provides a measure of how close the two sequences of shapes are. The total error will fall below a threshold when the guidewire 14 exits the clot C.

In other embodiments disclosed herein, a virtual representation of the guidewire tip 13 shape with a maximum curvature radius is overlaid on the live X-ray image 35 anchored to the current tip position. A user continuously compares the actual shape of the loop 17 with the maximum curvature shape acquired before entering the clot C.

In another example, the X-ray image 35 feedback can be enhanced with torque feedback to discriminate the “in-the-clot”/“out-of-the-clot” states. The robot 16 can be instrumented with force/torque sensors (not shown) that can measure the torque required to spin the guidewire 14. The apparatus 1 can measure the torque required to spin the guidewire 14 before the insertion and this can serve as a baseline value or baseline torque profile. Then the required torque can be continuously analysed as the guidewire 14 is advanced. While the guidewire 14 is in the clot C, the torque will be higher and there will be a drop as the guidewire 14 exits the clot C. This information can supplement the X-ray images 35 to improve the clot length detection.

In another embodiment, an alternative process includes classifying if a particular guidewire motion indicates being inside or outside of the clot C uses a common Convolutional Neural Network (CNN) implemented in the at least one electronic processor 20 of the electronic processing device 18. The input to the CNN is a sequence of X-ray images 35 that capture a full rotation (a partial rotation is also possible but not as reliable) along with a baseline sequence of rotation of the guidewire 14 prior to entry into the clot C. The output of the CNN is a binary classifier with probability of the two states, “in” or “out” of the clot C. The CNN is trained for each type of guidewire 14, and vessel diameter of the vessel V. In another example, feature engineering can be implemented, where the guidewire 14 is used for input as a sequence of normalized positions in the 2D images 35. The advantage of this approach is that it considers the full shape of the guidewire 14 instead of only the maximum radius of curvature.

In another embodiment, the robot 16 is configured to manipulate the guidewire 14 and retrieval devices 12, or as a hand-held device that helps with the traversing and characterization of the clot C. In the case of a hand-held device, the endo-vascular surgeon would navigate the retrieval device 12/guidewire 14 to the clot C and attach the hand-held device to the retrieval device 12/guidewire 14. The device can advance the guidewire 14 through the clot C using X-ray and/or force/torque feedback. Afterwards, the retrieval device 12 is advanced over the guidewire 14.

In another embodiment, the tip 13 of the guidewire 14 includes multiple compliant “tails” that increase the sensitivity. Each tail can have different mechanical properties such as stiffness, or friction coating.

These approaches can be used as well in other fields such as lead extraction and chronic total occlusion (CTO). The application is developed using X-ray images obtained from a C-arm like device. However, same concepts can be implemented using other imaging modalities such as CT, MRI, US, etc.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A thrombectomy device, comprising:
a retrieval device configured to deploy and subsequently retrieve an associated tethered self-expanding stent, the retrieval device including a guidewire having a tip that is radiopaque; and
at least one electronic processor programmed to:
receive a time sequence of images of extension of the guidewire through a clot during a thrombectomy procedure being performed using the thrombectomy device;
perform image analysis on the images of the time sequence of images to determine a geometric change of the tip of the guidewire;
identify a completion of the extension of the guidewire through the clot based on the geometric change of the tip of the guidewire; and
respond to the identification of the completion of the extension of the guidewire through the clot by:
outputting an indication that the guidewire is extended completely through the clot; and/or
controlling a robot to stop the extension of the guidewire through the clot.

2. The thrombectomy device of claim 1, wherein the image analysis determines the geometric change of the tip of the guidewire comprising a loop at the tip of the guidewire expanding after completion of the extension of the guidewire through the clot.

3. The thrombectomy device of claim 2, wherein the geometric change of the tip of the guidewire comprises the loop at the tip that is compressed during the extension of the guidewire through the clot and is expanded after completion of the extension of the guidewire through the clot.

4. The thrombectomy device of claim 1, wherein the at least one electronic processor is programmed to respond to the identification by outputting an indication that the guidewire is extended completely through the clot.

5. The thrombectomy device of claim 1, further comprising:
a robot controlled by the at least one electronic processor to automatically perform the extension of the guidewire through the clot.

6. The thrombectomy device of claim 1, further comprising:
an imaging device configured to acquire the time sequence of images of the guidewire acquired during the thrombectomy procedure;
wherein the imaging device is in communication with the at least one electronic processor.

7. The thrombectomy device of claim 6, wherein the imaging device comprises an X-ray imaging device including an X-ray source and an X-ray detector.

8. The thrombectomy device of claim 1, wherein the tip that is radiopaque comprises one or more radiopaque markers disposed on the tip.

9. The thrombectomy device of claim 1, wherein the tip that is radiopaque comprises a radiopaque wire disposed at the tip of the guidewire.

10. The thrombectomy device of claim 1, further including:
a sensor attached to the tip of the guidewire and configured to measure a contraction or an expansion of the tip.

11. A thrombectomy device, comprising:
a microcatheter;
a self-expanding stent tethered to the microcatheter and configured to be compacted into the microcatheter; and
a guidewire having a tip; and
wherein the tip of the guidewire is radiopaque and/or includes a sensor measuring a geometry of the tip.

12. The thrombectomy device of claim 11, wherein the thrombectomy device is configured to perform a thrombectomy procedure including:
deploying the guidewire through a clot;
extending the microcatheter with the self-expanding stent compacted into the microcatheter along the guidewire into the clot;
deploying the self-expanding stent by drawing back the microcatheter; and
retrieving the deployed self-expanding stent via the microcatheter to which the self-expanding stent is tethered.

13. The thrombectomy device of claim 11, wherein the tip of the guidewire is radiopaque and comprises a radiopaque wire and/or radiopaque markers disposed on the tip.

14. The thrombectomy device of claim 11, wherein the tip of the guidewire includes a bend sensor.

15. A vascular therapy method, comprising:
receiving a time sequence of images of extension of a guidewire through a clot during a thrombectomy procedure being performed using a thrombectomy device;
performing image analysis on the images of the time sequence of images to determine a geometric change of a tip of the guidewire;
identifying a completion of the extension of the guidewire through the clot based on the geometric change of the tip of the guidewire; and
responding to the identification of the completion of the extension of the guidewire through the clot by:
outputting an indication that the guidewire is extended completely through the clot; and/or
controlling a robot to stop the extension of the guidewire through the clot.

16. The vascular therapy method of claim 15, further comprising:
after identifying the completion of the extension of the guidewire through the clot, deploying a self-expanding stent along the guidewire and subsequently retrieving the self-expanding stent.

17. The vascular therapy method of claim 15, wherein the image analysis determines the geometric change of the tip of the guidewire comprising a loop at the tip of the guidewire expanding after completion of the extension of the guidewire through the clot.

18. The vascular therapy method of claim 15, wherein the geometric change of the tip of the guidewire comprises the loop at the tip that is compressed during the extension of the guidewire through the clot and is expanded after completion of the extension of the guidewire through the clot.

19. The vascular therapy method of claim 15, wherein the responding includes:
outputting an indication that the guidewire is extended completely through the clot.

20. The vascular therapy method of claim 15, wherein the responding includes:
controlling the robot to automatically perform the extension of the guidewire through the clot.
